(19) United States
(12) Patent Application Publication
(10) Pub. No.: US 2004/0244793 A1
(43) Pub. Date: Dec. 9, 2004

(54) Aroma inhaling product

(76) Inventor: Victor J. Wedel, Laguna Hills, CA (US)

Correspondence Address: RICHARD E. BEE, P.O. BOX 10544, COSTA MESA, CA, 92627, US

(21) Appl. No.: 10/457,278

(22) Filed: Jun. 9, 2003

(51) Int. Cl. A61M 16/00

(52) U.S. Cl. 128/200.24

(57) ABSTRACT

An aroma inhaling product comprising a frangible ampule and a plurality of essential oils having different vaporizing energies contained within the frangible ampule.

### BACKGROUND OF THE INVENTION

This invention relates to aroma inhaling products for enhancing moods and feelings of well-being with essential oils through the sense of smell by inhalation of the volatile vapors given off by such essential oils. Essential oils are obtained from plant and herbal materials and animal secretions. A common method of obtaining an essential oil is by steam or steam and water distillation by heating the plant or animal material in a water mixture and condensing the resulting vapor.

The use of the essential oils of flowers and plants stimulates and uplifts our spirits. When you feel melancholy or lethargic, for example, the simple inhalation of Jasmine or Orange blossom essential oil vapor tends to enhance your spirit. The direct inhalation of the odors generally affects the mind psychologically, although inhalation can also have physiological effect via the limbic system in the brain and the blood circulation.

It is desirable to have a small, compact and readily portable mechanism for transporting and easily using a premixed blend of essential oils. Such a mechanism is provided by an ampule type inhalant dispenser. As is known, such dispensers employ a frangible glass ampule containing a small quantity of a volatile inhalant. A crushing or breaking of the ampule with the fingers releases the volatile vapor for inhalation by a person in need of the stimulant. Such dispensers have been used for releasing ammonia vapors (smelling salts) for reviving persons who have fainted or lost consciousness.

Ampule type inhalant dispensers are ideally suited for use with essential oils to provide improved aroma inhaling products.

### SUMMARY OF THE INVENTION

The present invention provides unique and synergistic combinations of essential oils for enhancing human spirits in a variety of situations. The selection of essential oils which are combined is optimized to provide a harmonious blending of individual components to obtain improved results when the vapors are inhaled. The use of small, crushable ampules enables these blends to be premixed and easily and conveniently inhaled by a user at almost any time and place in which he or she may find himself or herself.

In one embodiment of the invention, an aroma inhaling product includes a frangible ampule. The product further includes a plurality of essential oils having different vaporizing energies contained within the frangible ampule.

In another embodiment, the aroma inhaling product includes a frangible ampule. The product also includes a first essential oil contained within the frangible ampule. The product further includes a second essential oil having a faster-acting vaporizing characteristic contained within the frangible ampule.

In a further embodiment of the invention, the aroma inhaling product includes a frangible ampule. The product also includes a first essential oil contained within the frangible ampule. The product further includes a second essential oil contained within the frangible ampule and having a shorter interval vaporizing characteristic than that of the first essential oil. The product also includes a third essential oil contained within the frangible ampule and having a shorter interval vaporizing characteristic than that of the second essential oil.

For a better understanding of the present invention, together with other and further advantages and features thereof, reference is made to the following description taken in connection with the accompanying drawings, the scope of the invention being pointed out in the appended claims.

### BRIEF DESCRIPTION OF THE DRAWINGS

Referring to the drawings:

FIG. 1 is a plan view of a representative embodiment of an aroma inhaling product constructed in accordance with the present invention; and

FIG. 2 is an enlarged cross-sectional view taken along section line 2-2 of FIG. 1.

### DETAILED DESCRIPTION OF THE ILLUSTRATED EMBODIMENT

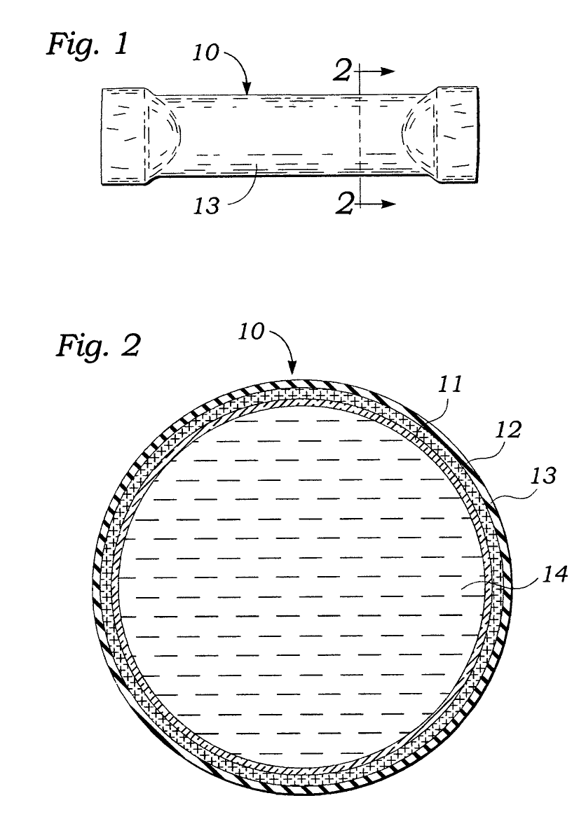

Referring to FIGS. 1 and 2, there is shown an aroma inhaling product 10 having a frangible glass ampule 11. A layer of protective material 12 surrounds the frangible ampule 11 for protecting the user's fingers when breaking the ampule 11. A vapor permeable outer casing 13 covers the protective material 12. When the ampule 111 is crushed or broken by pressure from the fingers of the user, the volatile liquid contained therein soaks into the protective material 12 to emit it's vapors through the vapor permeable outer casing 13.

The unbroken ampule 11 is filled with a pre-measured blend of ingredients 14. These ingredients 14 include a plurality of essential oils. Also contained within the ampule 11 may be a carrier oil for supplementing and providing a base material for at least one of the essential oils. This carrier oil may be, for example, Jojoba oil. A small quantity of alcohol may also be contained within the ampule 11 for providing a volatile carrier for the essential oils.

In accordance with the present invention, the different ones of the essential oils in ampule 11 are selected to have different vaporizing energies. In particular, a second one of the essential oils is selected to have a faster-acting vaporizing characteristic than that of a first one of the essential oils. For the case of three essential oils, the third essential oil is selected to have a faster-acting vaporizing characteristic than that of the second essential oil. In terms of time intervals, the second essential oil is selected to have a shorter interval vaporizing characteristic than that of the first essential oil and the third essential oil is selected to have a shorter interval vaporizing characteristic than that of the second essential oil. This distribution of vaporizing characteristics is selected to optimize the effectiveness of the stimulants for the different phases of the particular mood or feeling being considered.

The present invention is not limited to the use of herbal based essential oils. Animal based essential oils may also be used. Human phermones are a good example of an animal based essential oil.

An example of a combination of ingredients 14 that has found to be useful as a relaxant includes: Clary Sage, Tangerine, Lavender, and Jojoba oils and a small quantity of alcohol. A combination suitable for promoting alertness includes: Orange, Peppermint, Eucliptus, Lemongrass, and Jojoba oils and a small quantity of alcohol.

While there has been described what are at present considered to be preferred embodiments of this invention, it will be obvious to those skilled in the art that various changes and modifications may be made therein without departing from the invention and it is, therefore, intended to cover all such changes and modifications as come within the true spirit and scope of the invention.

What is claimed is:

1. An aroma inhaling product comprising:
a frangible ampule;
and a plurality of essential oils having different vaporizing energies contained within the frangible ampule.

2. An aroma inhaling product in accordance with claim 1 wherein a carrier oil is contained within the frangible ampule for supplementing and providing a base material for at least one of the essential oils.

3. An aroma inhaling product in accordance with claim 1 wherein a small quantity of alcohol is contained within the frangible ampule for providing a volatile carrier for the essential oils.

4. An aroma inhaling product in accordance with claim 1 wherein at least one of the essential oils is a phermone.

5. An aroma inhaling product in accordance with claim 1 and including protective material surrounding the frangible ampule for protecting the user when breaking the frangible ampule.

6. An aroma inhaling product in accordance with claim 5 and including a vapor permeable outer casing covering the protective material.

7. An aroma inhaling product comprising:
a frangible ampule;
a first essential oil contained within the frangible ampule;
and a second essential oil having a faster acting vaporizing characteristic contained within the frangible ampule.

8. An aroma inhaling product in accordance with claim 7 wherein a carrier oil is contained within the frangible ampule for supplementing and providing a base material for at least one of the essential oils.

9. An aroma inhaling product in accordance with claim 7 wherein a small quantity of alcohol is contained within the frangible ampule for providing a volatile carrier for the essential oils.

10. An aroma inhaling product in accordance with claim 7 wherein at least one of the essential oils is a phermone.

11. An aroma inhaling product in accordance with claim 7 and including protective material surrounding the frangible ampule for protecting the user when breaking the frangible ampule.

12. An aroma inhaling product in accordance with claim 11 and including a vapor permeable outer casing covering the protective material.

13. An aroma inhaling product comprising:
a frangible ampule;
a first essential oil contained within the frangible ampule;
a second essential oil contained within the frangible ampule and having a vaporizing characteristic which is faster acting than that of the first essential oil;
and a third essential oil contained within the frangible ampule and having a vaporizing characteristic which is faster acting than that of the second essential oil.

14. An aroma inhaling product in accordance with claim 13 wherein a carrier oil is contained within the frangible ampule for supplementing and providing a base material for at least one of the essential oils.

15. An aroma inhaling product in accordance with claim 13 wherein a small quantity of alcohol is contained within the frangible ampule for providing a volatile carrier for the essential oils.

16. An aroma inhaling product in accordance with claim 13 and including protective material surrounding the frangible ampule for protecting the user when breaking the frangible ampule.

17. An aroma inhaling product in accordance with claim 16 and including a vapor permeable outer casing covering the protective material.

18. An aroma inhaling product comprising:
a frangible ampule;
a first essential oil contained within the frangible ampule;
a second essential oil contained within the frangible ampule and having a shorter interval vaporizing characteristic than that of the first essential oil;
and a third essential oil contained within the frangible ampule and having a shorter interval vaporizing characteristic than that of the second essential oil.

19. An aroma inhaling product in accordance with claim 18 wherein a carrier oil is contained within the frangible ampule for supplementing and providing a base material for at least one of the essential oils.

20. An aroma inhaling product in accordance with claim 18 wherein a small quantity of alcohol is contained within the frangible ampule for providing a volatile carrier for the essential oils.

21. An aroma inhaling product in accordance with claim 18 and including protective material surrounding the frangible ampule for protecting the user when breaking the frangible ampule.

22. An aroma inhaling product in accordance with claim 21 and including a vapor permeable outer casing covering the protective material.
